(19) United States
(12) Patent Application Publication
(10) Pub. No.: US 2012/0296199 A1
(43) Pub. Date: Nov. 22, 2012

(54) Apparatus and Method of Non-Contrast Magnetic Resonance Angiography of Abdominal and Pelvic Arteries

(75) Inventors: Daniel KIM, Park City, UT (US); Vivan LEE, Salt Lake City, UT (US); Iliyana ATANASOVA, New York, NY (US)

(73) Assignee: New York University, New York, NY (US)

(21) Appl. No.: 13/426,171

(22) Filed: Mar. 21, 2012

Related U.S. Application Data
(60) Provisional application No. 61/454,832, filed on Mar. 21, 2011.

(51) Int. Cl. A61B 5/055

(52) U.S. Cl. 600/419

(57) ABSTRACT

Exemplary method and apparatus can be provided for generating information regarding at least one tissue. Using such exemplary method and apparatus, it is possible to generate a plurality of inversion recovery (IR) radio frequency (RF) pulses that are configured to establish an identifier of an element associated with at least one portion of the tissue(s). It is also possible to determine an inversion time associated with at least one of the RF pulses. Further, it is possible to generate, e.g., with a computing arrangement, the information regarding the tissue(s) based on the inversion time.

### CROSS-REFERENCE TO RELATED APPLICATION(S)

The present application relates to and claims priority from U.S. Provisional Patent Application No. 61/454,832 filed Mar. 21, 2011, the entire disclosure of which is incorporated herewith by reference.

### STATEMENT REGARDING FEDERALLY SPONSORED RESEARCH

The subject matter of the present disclosure was developed, at least in part, using Government support under Grant No. HL092439, awarded by the National Institute of Health and Grant No. 0730143N, awarded by the American Heart Association. Accordingly, the Federal Government has certain rights in the subject disclosure.

### FIELD OF THE DISCLOSURE

Exemplary embodiments of the present disclosure relate to medical imaging, and more specifically to method, system, and apparatus for providing non-contrast magnetic resonance angiography (MRA).

### BACKGROUND INFORMATION

Non-invasive assessment of the aortoiliac and the renal arteries can be used for the management of renal artery stenosis, and inflow peripheral arterial disease (PAD). Contrast-enhanced MR angiography (CE MRA) is a non-invasive procedure for evaluating vascular pathology in the abdomen and pelvis (See Prince M R. Gadolinium-enhanced MR aortography. Radiology 1994; 191(1):155-164; Hany T F, Debatin J F, Leung D A, Pfammatter T. Evaluation of the aortoiliac and renal arteries: comparison of breath-hold, contrast-enhanced, three-dimensional MR angiography with conventional catheter angiography. Radiology 1997; 204(2):357-362.). However, recent safety concerns associated with gadolinium-based contrast agents (See Grobner T, Prischl F C. Gadolinium and nephrogenic systemic fibrosis. Kidney Int 2007; 72(3):260-264.) have spurred a renewed interest in non-contrast MRA (NC MRA) techniques (See Miyazaki M, Lee V S. Nonenhanced MR angiography. Radiology 2008; 248(1):20-43; Miyazaki M, Takai H, Sugiura S, Wada H, Kuwahara R, Urata J. Peripheral MR angiography: separation of arteries from veins with flow-spoiled gradient pulses in electrocardiography-triggered three-dimensional half-Fourier fast spin-echo imaging. Radiology 2003; 227(3):890-896; Edelman R R, Sheehan J J, Dunkle E, Schindler N, Carr J, Koktzoglou I. Quiescentinterval single-shot unenhanced magnetic resonance angiography of peripheral vascular disease: Technical considerations and clinical feasibility. Magn Reson Med; 63(4):951-958; Katoh M, Buecker A, Stuber M, Gunther R W, Spuentrup E. Free-breathing renal MR angiography with steady-state free-precession (SSFP) and slab-selective spin inversion: initial results. Kidney Int 2004; 66(3):1272-1278). A number of abdominal NC MRA techniques rely on multiple pre-conditioning radio-frequency (RF) pulses for background suppression and inflow of unsaturated aortic blood for high vascular signal (Dixon W T, Sardashti M, Castillo M, Stomp G P. Multiple inversion recovery reduces static tissue signal in angiograms. Magn Reson Med 1991; 18(2):257-268; Mani S, Pauly J, Conolly S, Meyer C, Nishimura D. Background suppression with multiple inversion recovery nulling: applications to projective angiography. Magn Reson Med 1997; 37(6):898-905; Katoh M, Buecker A, Stuber M, Gunther R W, Spuentrup E. Free-breathing renal MR angiography with steady-state free-precession (SSFP) and slab-selective spin inversion: initial results. Kidney Int 2004; 66(3):1272-1278 Wyttenbach R, Braghetti A, Wyss M, Alerci M, Briner L, Santini P, Cozzi L, Di Valentino M, Katoh M, Marone C, Vock P, Gallino A. Renal artery assessment with nonenhanced steady-state free precession versus contrast-enhanced MR angiography. Radiology 2007; 245(1):186-195). NC MRA of the abdominopelvic arteries can be challenging due to a variety of factors including, e.g., respiratory motion suppression, large field of view (FOV) coverage, and depiction of vessels with variable hemodynamics and orientation. Fast spin-echo based subtraction techniques, that can rely on two acquisitions with differential flow sensitivities to arteries and veins can be useful for lower extremities (See Lim R P, Hecht E M, Xu J, et al. 3D nongadolinium-enhanced ECG-gated MRA of the distal lower extremities: preliminary clinical experience. J Magn Reson Imaging 2008; 28(1):181-189), but are likely not well-suited for the abdomen due to motion. Slice-selective (SS) inversion-recovery (IR) prepared “time-of-flight” based NC MRA with 3D b-SSFP readout has been developed for evaluation of renal arteries (See Katoh M, Buecker A, Stuber M, Gunther R W, Spuentrup E. Free-breathing renal MR angiography with steady-state free-precession (SSFP) and slab-selective spin inversion: initial results. Kidney Int 2004; 66(3):1272-1278) and validated in patients with renal artery stenosis (See Herborn C U, Watkins D M, Runge V M, Gendron J M, Montgomery M L, Naul L G. Renal arteries: comparison of steady-state free precession MR angiography and contrastenhanced MR angiography. Radiology 2006; 239(1):263-268; Maki J H, Wilson G J, Eubank W B, Glickerman D J, Millan J A, Hoogeveen R M. Navigator-gated MR angiography of the renal arteries: a potential screening tool for renal artery stenosis. AJR Am J Roentgenol 2007; 188(6):W540-546; Wyttenbach R, Braghetti A, Wyss M, et al. Renal artery assessment with nonenhanced steady-state free precession versus contrast-enhanced MR angiography. Radiology 2007; 245(1):186-195; Glockner J F, Takahashi N, Kawashima A, et al. Non-contrast renal artery MRA using an inflow inversion recovery steady state free precession technique (Inhance): comparison with 3D contrast-enhanced MRA. J Magn Reson Imaging 2010; 31(6):1411-1418) and renal transplant (See Liu X, Berg N, Sheehan J, et al. Renal transplant: nonenhanced renal MR angiography with magnetization-prepared steady-state free precession. Radiology 2009; 251(2):535-542). SS-IR b-SSFP MRA procedure can be typically performed axially with about 100-120 mm craniocaudal coverage, where a single SS IR radio-frequency (RF) pulse is applied transversely to overlay the imaging slab and extend inferiorly for venous suppression. The craniocaudal spatial coverage from renal arteries to distal external iliac arteries can be on the order of approximately 300 mm. One approach to extend the craniocaudal spatial coverage can be to perform coronal imaging with a thick IR pulse (See Shonai T, Takahashi T, Ikeguchi H, Miyazaki M, Amano K, Yui M. Improved arterial visibility using short-tau inversion-recovery (STIR) fat suppression in non-contrastenhanced time-spatial labeling inversion pulse (Time-SLIP) renal MR angiography (MRA). J Magn Reson Imaging 2009; 29(6):1471-1477), facilitating an extended visualization of the aorta. However, a single SS-IR b-SSFP MRA acquisition that can provide spatial coverage from renal arteries to iliac arteries with adequate background suppression has not been reported.

Accordingly, there may be a need or a benefit to address and/or overcome at least some of the issues and/or deficiencies described herein above.

### SUMMARY OF EXEMPLARY EMBODIMENTS

According to certain exemplary embodiments of the present disclosure, methods and apparatus can be provided which can facilitate/utilize a non-contrast MRA pulse sequence using 4 inversion-recovery (IR) pulses to provide coverage (e.g., a significant or complete coverage) from renal arteries to iliac arteries with a preferential background suppression. For example, the inversion times (TIs) and positions of the slice-selective IR pulses can be based on the Bloch equation governing T₁ relaxation, T₁ values of blood, tissue, and fat, and typical arterial blood flow rate. After pre-conditioning the magnetization with 4 IR pulses, a 3D b-SSFP readout can be used to image with bright arterial contrast.

For example, in particular exemplary embodiments of the present disclosure, methods and apparatus can be provided which can facilitate/utilize a preferential arterial conspicuity from the renal to the distal external iliac arteries with optimal background suppression. For example, both NC MRA and contrast-enhanced MRA can indicate a beneficial depiction of an infrarenal aortic atherosclerosis and left internal iliac artery origin stenosis.

According to further exemplary embodiments of the present disclosure, methods and apparatus can be provided which can facilitate/utilize a non-contrast MRA pulse sequence, which can provide high spatial resolution (e.g., 1.3 mm×1.3 mm×1.7 mm) visualization of abdominopelvic arteries within clinically feasible scan times of about 7 min.

Pursuant to another exemplary embodiment of the present disclosure, methods and apparatus can be provided which can facilitate/utilize an extended craniocaudal spatial coverage (e.g., renal to external iliac arteries) of IR b-SSFP MRA by utilizing four IR pulses. Certain additional exemplary embodiments of the present disclosure provide methods and apparatus which can facilitate/utilize a NC MRA pulse sequence for extended coverage of the abdominopelvic arteries and evaluations of its performance.

In yet another exemplary embodiment of the present disclosure, exemplary method and apparatus can be provided for generating information regarding at least one tissue. Using such exemplary method and apparatus, it is possible to generate a plurality of inversion recovery (IR) radio frequency (RF) pulses that are configured to establish an identifier of an element associated with at least one portion of the tissue(s). It is also possible to determine an inversion time associated with at least one of the RF pulses. Further, it is possible to generate, e.g., with a computing arrangement, the information regarding the tissue(s) based on the inversion time.

The tissue(s) can include an aortic bifurcation, and the element can include blood traveling through the aortic bifurcation. The RF pulses can include at least one first nonselective IR RF pulse configured to invert a longitudinal magnetization of at least one background tissue. The identifier can be established by re-inverting a longitudinal magnetization of the element associated with the tissue(s). The RF pulses can include a graphically oriented slice-selective RF pulse configured to re-invert the longitudinal magnetization of magnetic spins of the element associated with the tissue(s). A graphical orientation of the graphically oriented slice-selective RF pulse can be received, and the information can be generated using the graphical orientation. The graphical orientation can facilitate substantially focusing on the tissue(s) and exclude background tissues. The RF pulses can include a graphically oriented further slice-selective RF pulse configured to invert a longitudinal magnetization of an additional tissue. A graphical orientation of the graphically oriented further slice-selective RF pulse can be received, and the information can be generated using the graphical orientation of the further slice-selective RF pulse.

According to another exemplary embodiment of the present disclosure, the RF pulses can include a short inversion time inversion recovery (STIR) RF pulse configured to invert a longitudinal magnetization of the tissue(s)s within an imaging volume. The RF pulses can also include a graphically oriented slice-selective RF pulse configured to re-invert the longitudinal magnetization of magnetic spins of the element associated with the tissue(s) immediately following a first nonselective RF pulse which is configured to invert a longitudinal magnetization of background tissues. The tissue(s) can include an aortic bifurcation and the RF pulses can be IR pulses.

These and other objects, features and advantages of the exemplary embodiment of the present disclosure will become apparent upon reading the following detailed description of the exemplary embodiments of the present disclosure, along with the appended claims.

### BRIEF DESCRIPTION OF THE DRAWINGS

Further objects, features and advantages of the present disclosure will become apparent from the following detailed description taken in conjunction with the accompanying FIGs. showing illustrative embodiments of the present disclosure, in which:

FIG. 1A is an illustration of a pulse sequence diagram and exemplary images of a non-contrast magnetic resonance angiography generated using apparatus and method in accordance with an exemplary embodiment of the present disclosure;

FIG. 1B is an exemplary graph of a longitudinal magnetization as a function of time for background tissues after inversion-recovery pulses generated using the apparatus and method in accordance with an exemplary embodiment of the present disclosure;

FIGS. 2A-2F are exemplary images of left and right iliac arteries and veins at different inversion time settings generated using the apparatus and method according to exemplary embodiments of the present disclosure;

FIGS. 3A-3B are first sets of exemplary NC MRA images of an abdominopelvic angiograms generated using the apparatus and method according to further exemplary embodiments of the present disclosure;

FIGS. 4A-4F are second sets of exemplary NC MRA images of an abdominopelvic angiograms generated using the apparatus and method according to additional exemplary embodiments of the present disclosure;

FIGS. 5A-5C are third sets of exemplary NC MRA images of an abdominopelvic angiograms generated using the apparatus and method according to additional exemplary embodiments of the present disclosure;

FIG. 6A is another exemplary NC MRA image of an abdominopelvic angiograms generated using the apparatus and method according to additional exemplary embodiments of the present disclosure;

FIG. 6B is a CE MRA image of the same region shown in FIG. 6A; and

FIG. 7 is yet another an exemplary NC MRA image of an abdominopelvic angiograms generated using the apparatus and method according to additional exemplary embodiments of the present disclosure;

FIG. 8 is an exemplary block diagram of an exemplary embodiment of a system according to the present disclosure; and

FIG. 9 is a flow diagram illustrating an exemplary procedure according to exemplary embodiments of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described exemplary embodiments without departing from the true scope and spirit of the subject disclosure.

### DETAILED DESCRIPTION OF EXEMPLARY EMBODIMENTS

Exemplary embodiments of the apparatus and methods according to the present disclosure can provide, facilitate and/or generate a NC MRA pulse sequence for comprehensive assessment of abdominopelvic arteries, using a multiple number of IR pulses and 3D b-SSFP readout with respiratory triggering. Although certain exemplary embodiment of the apparatus and methods according to the present disclosure can utilize four IR pulses for imaging of the abdominopelvic arteries and is described herein in detail, other exemplary embodiments of the present disclosure can include the usage of other numbers of pulses, such as five pulses, six pulses, seven pulses, etc., and can be used to image arteries in other regions of the body. This exemplary pulse sequence can he an extension and/or an improvement of the previously described SS-IR b-SSFP MRA pulse sequence that was designed for renal arteries. One exemplary advantage of the exemplary NC MRA according to the present disclosure over the previous SS-IR b-SSFP MRA can be, e.g., that spatial coverage of arterial visualization was approximately doubled for a similar transmit time for arterial inflow. Exemplary NC MRA methods according to the present disclosure can provide coverage from renal arteries to the distal external iliac arteries with sufficient background suppression.

Such exemplary pulse sequence according to the present disclosure can have several interesting aspects. First, for patients with severe disease and slow flow, it may be preferable to increase the TI to achieve sufficient arterial coverage, at the expense of increasing the background signal. Second, the use of the exemplary SS reinversion pulse according to the present disclosure can generate a brighter background “band” where the pulse was applied that can lead to at least a partial visualization of pelvic organs (e.g., bladder). This band can be detected and removed during post processing. Third, a careful selection of the thickness and positioning of the reinversion pulse can be preferable to avoid an enhancement of background and veins. For careful planning, high resolution scout images may preferably be utilized according to one exemplary embodiment of the present disclosure. Fourth, the mean scan time of the acquisition can be on the order of, e.g., 7 minutes, which can be a clinically feasible scan time. To further accelerate the pulse sequence, acceleration methods such as compressed sensing (See Lustig M, Donoho D, Pauly J M. Sparse MRI: The application of compressed sensing for rapid MR imaging. Magn Reson Med 2007; 58(6):1182-1195) and highly accelerated parallel imaging (See Sodickson D K, Griswold M A, Jakob P M, Edelman R R, Manning W J. Signal-to-noise ratio and signal-to-noise efficiency in SMASH imaging. Magn Reson Med 1999; 41(5):1009-1022;1 Pruessmann K P, Weiger M, Scheidegger M B, Boesiger P. SENSE: sensitivity encoding for fast MRI. Magn Reson Med 1999; 42(5);952-962) with a 32-element coil array can be utilized, as well as synchronized breathing to increase respiratory triggering efficiency.

According to exemplary embodiments of the present disclosure, the exemplary apparatus and methods can provide facilitate an achievement of an arterial visibility from the suprarenal aorta to distal external iliac arteries with sufficient background suppression. For example, CR_{A-B}, which can be defined as the signal contrast (or difference) between the artery and background divided by the arterial signal, measurements can show that NC MRA exhibits good static background attenuation in both controls and patients, comparable to CR_{A-B} of source CE MRA. These results can reflect the efficacy of the IR preconditioning for background nulling. The STIR pulse can be essential for robust suppression of subcutaneous fat and short-T₁ intestinal contents. A STIR pulse can be selected instead of a chemical-shift selective RF pulse, which can be more sensitive to static field (B₀) and transmit RF field (B₁) inhomogeneities, especially given a large FOV. The STIR pulse combined with the b-SSFP readout with large excitation angles and linear k-space ordering can maintain adequate static tissue suppression even when TI was prolonged for extended coverage.

In another exemplary embodiment of the present disclosure, a venous suppression can also be achieved by, e.g., separate exemplary mechanisms and/or procedures. For example, a signal from venous spins that remain within the FOV throughout the entire experiment (i.e., venous blood that originally occupies the iliac veins and moves to the vena cava by the start of the readout) can be attenuated by the NS-IR and the STIR pulses. Additionally, a signal from inflowing venous blood, found primarily in the external and common iliac veins, can be governed by the inversion time of the caudal SS-IR pulse. An overall venous suppression can be adequate with external iliac veins exhibiting more favorable CR_{A-V}, which can be defined as the signal contrast between the artery and vein divided by the artery signal, and CR_{V-B}, which can be defined as the signal contrast between the vein and background divided by the venous signal, compared to other segments. This can be due to the reinversion effect of the sagittal SS-IR that frequently affected the vena cava and a portion of the common iliac veins.

A respiratory motion in the abdomen and pelvis can pose a challenge to the robustness of non-contrast MRA techniques. In prior renal NC MRA studies, motion artifacts have been controlled with navigator-gating, which has been reported to provide more robust motion suppression for liver and biliary tree assessment compared to bellows triggering. However, the reliability of navigator gating versus bellows triggering has not been compared in the context of abdominopelvic MRA. Furthermore, the liver-to-diaphragm interface, where the navigator beam is typically positioned, can be situated far from the magnet isocenter. Consequently, it may be difficult to accurately detect the diaphragmatic position for abdominopelvic NC MRA.

According to certain exemplary embodiments of the present disclosure, no significant motion degradation of image quality may be observed using bellows triggering. Respiratory-triggered acquisitions, however, can lead to prolonged scan times in subjects with low breathing rates. In these cases, it an be helpful to accelerate the pulse sequence using more advanced view ordering schemes or highly-accelerated parallel imaging, for example, with a 32-element coil array at the expense of SNR or spatial resolution, and/or use synchronized breathing to increase the respiratory triggering efficiency.

This exemplary procedure can have several challenging aspects. For example, as the craniocaudal coverage beyond the aortic bifurcation is typically flow-dependent, insufficient conspicuity of the iliac arteries may be anticipated in patients with severely slow flow. In general, the coverage may be extended by prolonging TI at the expense of increased background signal. Indicators of reduced cardiac output (e.g., hypotension, cardiac failure), monitoring heart rate, measuring arterial flow velocity using phase contrast MRI, or performing a rapid, low-resolution version of the exemplary NC MRA as a “scout” pulse sequence to estimate the coverage of arterial visualization prior to NC MRA can guide a selection of an appropriate TI and ensure robustness of the technique across a wide spectrum of vascular conditions and blood flow rates.

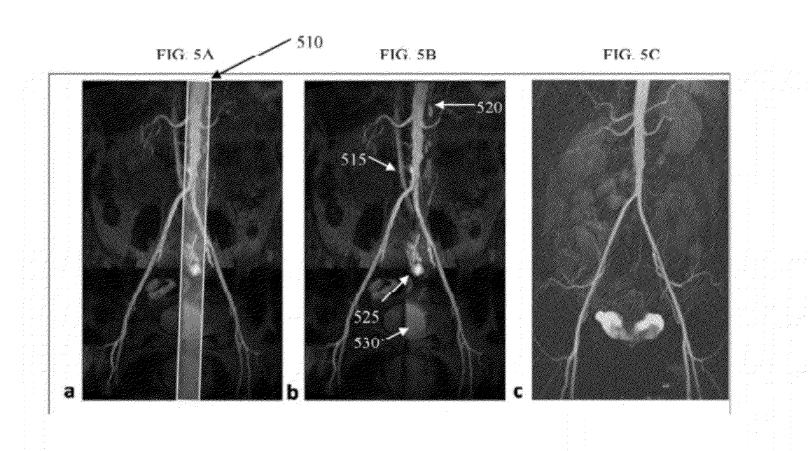

Further, it can be preferable to facilitate that the sagittal SS-IR pulse reinverts the entire volume of the abdominal aorta. Failure to do so can reduce an arterial visibility distal to the aortic bifurcation. Obtaining scout images of the entire abdominal aorta with coverage up to the heart can therefore be preferable for careful planning. The SS-IR pulse can also result in a brighter background sagittal“band’ that can lead to a partial visualization of pelvic organs (see FIGS. 5A-5C). Although this band typically does not degrade diagnostic quality, minimizing the width of the SS-IR pulse to avoid unnecessary enhancement of background and veins is preferred.

Additionally, the b-SSFP readout can be prone to off-resonance artifacts. Severe signal loss caused by bowel gas can occur in two arterial segments. Reduced signal intensity can be frequently observed in the suprarenal aorta, likely due to rapid blood flow through an off-resonance region, given that during initial technical optimization of b-SSFP parameters, this signal loss can be observed to deteriorate for longer inter-echo spacing. Complete signal voids can be prevented by shortening the excitation pulse duration and increasing the receiver bandwidth to achieve a short TR of 3.3 ms. The artifact can be further reduced with ECG-triggering by acquiring the image during diastole. However, the combined use of ECG-triggering and respiratory gating can significantly prolong the scan time, and can be impractical for clinical imaging. Previous inflow-based MRA studies (7-10) have utilized ECG-triggering to include at least one systolic period prior to data acquisition. TI can be longer than a typical cardiac cycle and, therefore, provide one systolic period prior to data acquisition.

According to certain exemplary embodiments of the present disclosure, exemplary apparatus and methods can be provided which can facilitate, utilize and/or generate an exemplary NC MRA pulse sequence using a plurality of (e.g., four) IR pulses with 3D b-SSFP readout for imaging of abdominopelvic arteries. Such exemplary NC MRA can provide high spatial resolution visualization of the aortoiliac and renal arteries with comprehensive head-to-foot coverage and excellent background and venous suppression in clinically feasible scan times.

### Exemplary Experiment

### Exemplary Materials and Methods

### Exemplary Blood Flow Velocity Measurements

Described below is an exemplary experiment implementing and/or utilizing certain exemplary embodiments of the present disclosure. Indeed, the exemplary embodiments of the present disclosure are in no way limited to the exemplary experiment described below, and other implementations of embodiments of the present disclosure are contemplated, as should be understood by those having ordinary skill in the art reading the present disclosure. The spatial coverage of arterial inflow and magnitude of background longitudinal magnetization (M_{z}) can be governed by TI and arterial blood flow rate. Accordingly, a longer TI can extend the coverage at the cost of increased background signal, and vice versa. In an exemplary experiment implementing and/or utilizing the exemplary embodiments of the present disclosure, e.g., to optimize the pulse sequence for sufficient arterial inflow coverage, arterial velocity measurements were obtained with phase contrast MRI in the distal infrarenal aorta, for example, of 14 patients (7 female, 7 male; mean age, 65.3 year; age range 29-93). Eleven of the subjects, for example, were referred for chest MRA for suspected aortic aneurysm or dissection. In three patients, for example, peripheral MRA was requested for suspected PAD. The measurements can be performed with phase contrast MRI with electrocardiogram gated phase contrast MRI (2D FLASH, TR 65.3 ms, TE 5.07 ms, FA 20°, TA 18-24 sec, through-plane velocity encoding 100-150 cm/s). Flow velocities can be measured, for example, using Siemens Argus software with regions of interest drawn to encompass the entire vessel lumen, preferably excluding the vessel wall. Reported values can represent average arterial velocities over one cardiac cycle.

In an exemplary embodiment of the present disclosure, individual patient measurements can be averaged to calculate mean arterial velocity, V_{avg}, across the subjects. To obtain arterial visibility from the renal to the distal iliac arteries, the entire length of the iliac arteries, which measure on the order of 200 mm, is preferably filled with unsaturated arterial blood. Assuming constant arterial velocity, a rough estimate of TI for sufficient coverage was calculated, for example, by TI=200 mm/V_{avg}.

In the exemplary preliminary study of 14 patients (e.g., 5 without pathology, 1 with dissecting descending aorta, 3 with infrarenal aneurysm, 3 with enlarged thoracic aorta, 2 with iliac stenosis), presence and severity of disease can vary considerably, resulting in a wide range of measured velocities, varying from 4.7 to 23.2 cm/s, with V_{avg}=13.8±4.8 cm/s. To calculate TI appropriate for volunteers without pathology, two patients whose measurements were obtained within abdominal aortic aneurysms can be excluded from analysis, resulting in V_{avg}=15.2±3.1 cm/s and TI for suitable coverage of approximately 1300 ms. For subsequent in vivo NC MRA imaging, TI can be set to 1300 ms when no disease was suspected and was prolonged by 400 ms when indications for reduced flow rates were present.

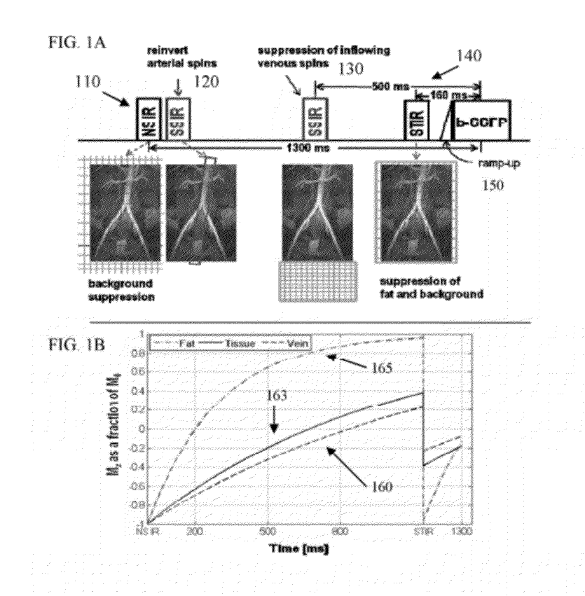

To examine whether the TI values identified for sufficient coverage should theoretically provide adequate background suppression, M_{z} at the center of k-space can be calculated using the Bloch equation governing T₁ relaxation for three types of background (veins, tissue, and fat). Exemplary graphs (e.g., see FIG. 1B) of M_{z} as a function of time after IR pulses demonstrate a near complete suppression of background signals at the center of k-space with TI=1300 ms. When TI is prolonged to 1700 ms (plot not shown), background M_{z} can increase, for example, compared to the 1300 ms case as follows: venous M_{z} from 0.07M₀ to 0.27M₀, tissue M_{z} from 0.17M₀ to 0.35M₀, and fat M_{z} from 0.13M₀ to 0.14M₀. These values can imply that background signal will be brighter with TI=1700 ms, but will remain sufficiently low for satisfactory arterial conspicuity. The exemplary results can be calculated assuming inversion of magnetization by IR pulses, ideal slice profile of SS-IR, and tissue T₁ 966 ms, fat T₁ 288 ms, and venous T₁ 1200 ms.

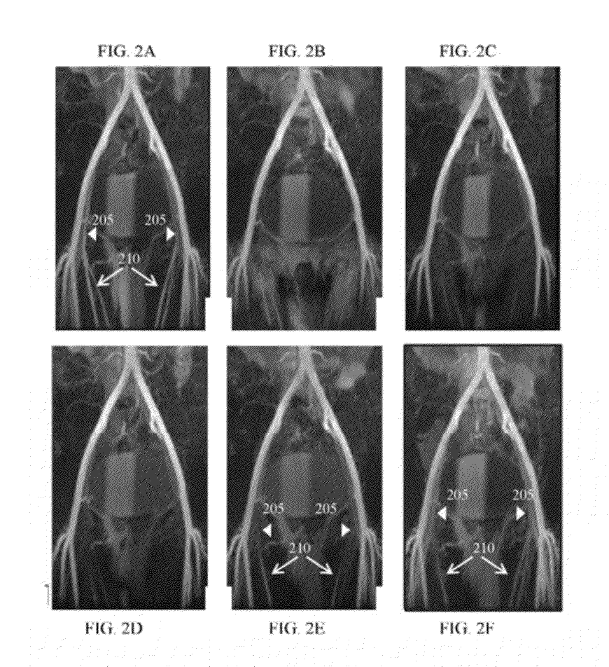

Given the unknown efficacy of the IR pulses outside the FOV, a range of TIᵥₑₙₒᵤₛ values can be tested, for example, in two volunteers (2 male, ages 30 and 49) to empirically determine TIᵥₑₙₒᵤₛ for optimal suppression of inflowing venous blood from femoral veins. NC MRA can be performed with the following settings for TIᵥₑₙₒᵤₛ of the caudal SS-IR pulse: none, 300 ms, 500 ms, 700 ms, 900 ms, and 1100 ms. Signal intensity of the iliac veins can be assessed to determine TIᵥₑₙₒᵤₛ yielding minimal venous signal. TIᵥₑₙₒᵤₛ of 500 ms provided best suppression (e.g., see FIG. 2C) and was used for subsequent in vivo NC MRA imaging.

### Exemplary Quadruple IR Pulse

The exemplary quadruple IR pulse scheme can facilitate, e.g.; (i) craniocaudal coverage of the abdominopelvic arteries from suprarenal aorta to distal iliac arteries and (ii) adequate background suppression. FIG. 1A illustrates an exemplary quadruple IR pulse scheme and associated images in accordance with exemplary embodiments of the present disclosure. First, at 110, a non-selective (NS) IR pulse can be applied with inversion time TI to invert the M_{z} of tissues and attenuate the background. Second, after the NS IR pulse, a SS IR pulse overlaying the aorta in a sagittal plane can be applied, at 120, to re-invert the spins in the aortic blood, from top of the FOV to aortic bifurcation. At 130, a SS-IR pulse with inversion time TIᵥₑₙₒᵤₛ can be applied caudal to the imaging FOV for suppression of inflowing venous spins. Lastly, at 140, a short tau IR (STIR) pulse with, e.g., TI 160 ms prior to 3D b-SSFP readout to suppress fat and further attenuate the background can be applied. Ten linearly increasing ramp-up excitation pulses can be used, e.g., at 150, to accelerate the approach to steady state of magnetization. Exemplary imaging can be performed (e.g., using a computing arrangement) with an oblique coronal 3D b-SSFP readout with 90° excitation RF pulses and a linear k-space ordering to further attenuate background signals.

During the interval TI the M_{z} of the background can recover, while inverted aortic blood can be replaced with fully-magnetized inflowing spins. An exemplary selection of TI is preferably a balance between arterial inflow and background suppression; longer TI can extend coverage at the cost of increased background signal. The combination of NS-IR and sagittal SS-IR pulses can decrease the time necessary to obtain coverage from renal to distal external iliac arteries by “pre-filling” fresh arterial spins up to aortic bifurcation. Coverage beyond the bifurcation is typically governed by TI and the flow rate of arterial blood.

The M_{z} of static background tissues (e.g., fat, muscle, vein) within the FOV, but outside the sagittal SS-IR, can undergo inversion recovery prior to the application of the STIR pulse with TI of 160 ms. Thus, for each tissue type, its M_{z} can be calculated as a function of TI of the NS-IR pulse using the Bloch equations governing T₁ relaxation. Background spins within the sagittal SS-IR excitation slab can be reinverted to an equilibrium magnetization, and these can be attenuated by the b-SSFP readout with large excitation angles and linear k-space ordering, and can be further removed manually during post processing.

To suppress the signal from inflowing iliac venous blood, after the NS-IR pulse and prior to the STIR pulse, a second SS-IR pulse graphically positioned in axial orientation can be used, caudal to the FOV. The combined use of this IR pulse with TIᵥₑₙₒᵤₛ and STIR pulse with TI 160 ms can achieve effective suppression of inflowing venous blood.

According to an exemplary embodiment of the present disclosure, TI can be selected to achieve an appropriate balance between sufficient coverage of arterial inflow and background suppression. Assuming arterial velocity of about 14 cm/s (see results), a selection of a TI of approximately 1300 ms predicts approximately that magnetized signal from the aorta will travel 180 mm beyond the aortic bifurcation. This exemplary distance can be sufficient for visualization of the length of the iliac arteries.

FIG. 1B shows an exemplary graph of M_{z} as a function of time after IR pulses for three types of background tissues: veins 160, tissue 163, and fat 165. These exemplary results can be calculated or determined using, e.g., the Bloch equation governing T₁ relaxation, assuming inversion of magnetization by IR pulses, ideal slice profile of SS-IR, and tissue T₁ 966 ms (See de Bazelaire C M, Duhamel G D, Rofsky N M, Alsop D C. MR imaging relaxation times of abdominal and pelvic tissues measured in vivo at 3.0 T: preliminary results. Radiology 2004; 230(3):652-659), fat T₁ 288 ms (See Gold G E, Han E, Stainsby J, Wright G, Brittain J, Beaulieu C. Musculoskeletal MRI at 3.0 T: relaxation times and image contrast. AJR Am J Roentgenol 2004; 183(4343-351.), and venous T₁ 1200 ms (See Parker D L, Tsuruda J S, Goodrich K C, Alexander A L, Buswell H R. Contrast-enhanced magnetic resonance angiography of cerebral arteries. A review. Invest Radiol 1998; 33(9):560-572).

A NS-IR pulse can be applied for background suppression. Subsequently, the aorta can be reinverted by a SS-IR pulse graphically prescribed sagittally. A third SS-IR pulse with inversion time TIᵥₑₙₒᵤₛ can be graphically applied inferiorly to the imaging FOV for suppression of inflowing venous blood. A STIR pulse can be applied with TI of 160 ms prior to acquisition for fat suppression and additional attenuation of background tissues. The b-SSFP readout can be preceded by 10 linearly increasing excitation pulses (ramp-up) to accelerate the approach to steady-state of magnetization. The 3D b-SSFP imaging can be performed with large flip angles and linear k-space ordering to further attenuate background tissues which have relatively low T₂. The center of k-space can be acquired a time interval TI after to the first inversion. Mz can be calculated or determined for the background outside the sagittally oriented IR slab, which may not experience the first SS IR 120, and may not be excited by the second SS-IR 130. The choice of inversion parameters can facilitate a near complete suppression of background signals at the center of k-space. For example, background spins within the sagittal SS IR slab can appear as a ‘band’ of bright signal, e.g., with settings 76×127 mm (600×600 DPI).

The TIᵥₑₙₒᵤₛ value can be empirically derived from exemplary images of left and right iliac arteries and veins at different inversion time settings in generated using the apparatus and method according to exemplary embodiments of the present disclosure shown in FIGS. 2(a)-2(f), since it can be challenging to model M_{z} of inflowing venous spins without knowing the true flip angles outside the FOV. The background is typically not excited by the second SS-IR pulse. After the exemplary application of the second SS-IR pulse, a STIR pulse can be applied to suppress and/or reduce fat and further attenuate background tissue and veins. Additional pulses can be applied to further attenuate background tissue and veins.

### Exemplary Human Studies

An exemplary study described herein was approved by the Institutional Review Board and informed consent was obtained from all subjects. In an exemplary experiment implementing exemplary embodiments of the present disclosure, an exemplary imaging procedure can be performed on a whole-body 1.5T system (Avanto, Siemens Healthcare, Erlangen, Germany) equipped with a gradient system capable of achieving preferential and/or maximum gradient strength of about 45 mT/m and a slew rate of about 200 T/m/s. The RF excitation can be performed, for example, using the body coil; two body coil arrays and a spine coil array were employed for signal reception. NC MRAs were obtained using the quadruple IR 3D b-SSFP oriented in an oblique coronal slab with respiratory bellows for triggering (e.g., 20% end expiration or 20% end inspiration).

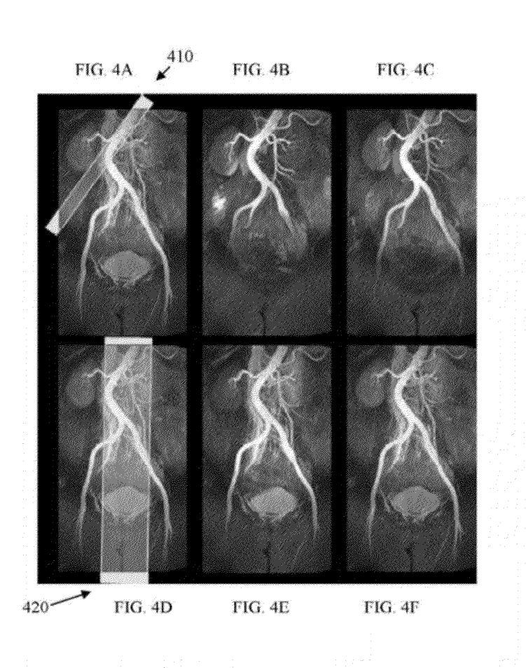

Imaging parameters included: FOV 400 mm×400 mm, 60-80 partitions, nominal slice thickness 1.7 mm, acquisition matrix=320×307, slice resolution 65%, phase oversampling 10%, slice oversampling 20-40%, spatial resolution 1.3×1.3×1.7 mm₃, TR 1 respiratory cycle, TE 1.7 ms, FA 90°, BW 1042 Hz/pixel, 60 k-space lines per shot, 2 shots per partition, GRAPPA (18) effective acceleration factor 2.7. IR parameters included: NS IR pulse with TI 1300 ms; 20-60 mm thick IR pulse oriented in oblique sagittal plane with TI of 1280 ms; 200 mm SS IR pulse positioned inferiorly to the FOV with TIᵥₑₙₒᵤₛ of 500 ms; STIR with TI 160 ms. Seven healthy volunteers (1 female, 6 male, age range 23-52; mean age 34) and 10 patients (4 female, 6 male; age range 57-85; mean age 73) can be imaged using the aforementioned protocol. Seven volunteers and four patients can be imaged with TI=1300 ms; in six patients TI=1700 ms can be used based on prior history of pathology (e.g., aneurysm, low cardiac output). To account for more complex geometries of the aorta, the effects of the positioning and thickness of the SS reinversion pulse were investigated in a female volunteer with known scoliosis (age 51). Images were obtained with TI=1300 ms for two different positions of the sagittal slab as displayed in FIGS. 4A and 4D. Imaging was repeated with TI=1500 ms to examine the influence of TI on arterial visibility and background suppression.

### Exemplary NC MRA

In an exemplary implementation/utilization according to certain exemplary embodiments of the present disclosure, a three-plane dark-blood Half-Fourier acquisition single-shot turbo spin echo (HASTE) scout scan can be used for image planning. Subsequently, NC MRA can be performed using an oblique coronal imaging slab, oriented along the abdominal aorta, with respiratory bellows for triggering (e.g., 20% end expiration or 20% end inspiration). Imaging parameters can include: TR 1 respiratory cycle, TE 1.7 ms, FA 90°, BW 1042 Hz/pixel, 60 k-space lines per shot, 2 shots per partition, generalized autocalibrating partially parallel acquisitions (GRAPPA) effective acceleration factor 2.7, FOV 400 mm×400 mm, 60-80 partitions, nominal slice thickness 1.7 mm, acquisition matrix=320×307, slice resolution 65%, phase oversampling 10%, slice oversampling 20-40%, spatial resolution 1.3 mm×1.3 mm×1.7 mm. IR parameters can include: NS IR pulse with TI between 1300 ms and 1700 ms as detailed below; 20-60 mm thick IR pulse oriented in oblique sagittal plane overlying the abdominal aorta (inversion time 1280 ms); 200 mm thick SS-IR pulse positioned axially immediately inferior to the FOV (TIᵥₑₙₒᵤₛ=500 ms); STIR with inversion time 160 ms.

All volunteers can undergo imaging using the aforementioned protocol. In addition, one of the control subjects (male, age 29) can be imaged with the sagittal SS-IR pulse turned off to illustrate the utility of the re-inversion pulse. The effect of the position and thickness of this pulse can be further investigated in a volunteer with known scoliosis (female, age 51). Exemplary imaging can be performed with TI=1300 ms for two positions of the sagittal slab and repeated with TI=1500 ms to illustrate the influence of TI on arterial visibility and background suppression. Four patients can be imaged with TI=1300 ms; in six patients TI=1700 ms can be used in view of indications of reduced flow rates (e.g., age≧70 years, known aneurysm).

### Exemplary Results

FIGS. 2A-2F show exemplary NC-MRAs images of left and right iliac arteries and veins at different inversion time TIᵥₑₙₒᵤₛ settings using the apparatus and method according to certain exemplary embodiments of the present disclosure. Among the different TIᵥₑₙₒᵤₛ values, in this exemplary embodiment, TIᵥₑₙₒᵤₛ of 500 ms achieved sufficient suppression of venous blood. This value may vary depending on the equipment and patient, and other values of TIᵥₑₙₒᵤₛ may be preferred for different systems and patients. FIGS. 2A to 2F illustrate exemplary maximum intensity projection of left and right iliac arteries and veins obtained in a volunteer. In particular, FIG. 2A shows an exemplary signal intensity of the right and left common iliac veins without a caudal SS-IR pulse. FIG. 2B to 2F illustrate an exemplary signal intensity of the right and left common iliac veins with a caudal SS-IR pulse setting of: TIₙᵤₗₗ 300 ms (2B), TIₙᵤₗₗ 500 ms (2C), TIₙᵤₗₗ 700 ms (2D), TIₙᵤₗₗ 900 ms (2E), and TIₙᵤₗₗ 1100 ms (2F), respectively. FIG. 2C illustrates a preferable suppression of inflowing venous blood. In this figure, arrowheads 205 denote exemplary external iliac veins, and arrows 210 point to exemplary branches of the femoral vein.

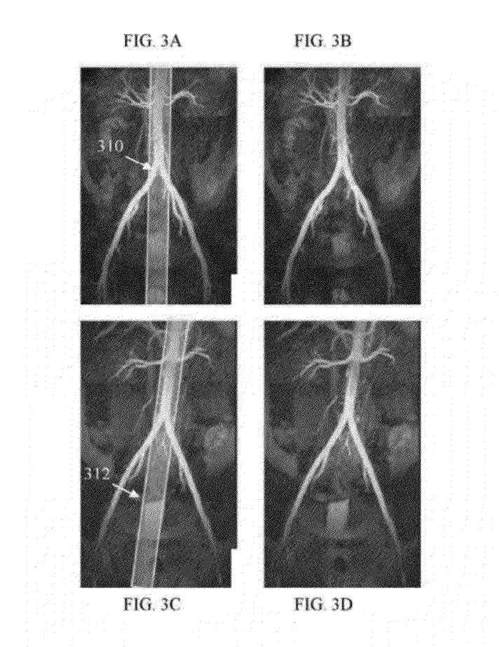

FIGS. 3A and 3B show exemplary angiograms of a 31 year old volunteer using the exemplary Sagittal SS-IR slab positioning 310 (e.g., in FIG. 3A), and an exemplary resultant NC MRA image using the sagittally oriented SS-IR pulse apparatus and method according to particular exemplary embodiments of the present disclosure (e.g., as shown in FIG. 3B). Exemplary images of FIGS. 3A and 3B illustrates a preferable arterial conspicuity with, e.g., comprehensive coverage and optimal background and venous suppression. As shown in FIGS. 3A and 3B, for example, the exemplary images demonstrate the utility of the rein version pulse to reduce the time typically needed to achieve complete filling fully magnetized arterial spins from renal to distal iliac arteries. With all other parameters kept the same, the application of the SS-IR pulse can increase craniocaudal coverage by approximately 170 mm without prolonging inflow time at the expense of brighter background (arrows) coincident with the location of the rein version pulse. Both images were acquired with about TI=1300 ms. FIGS. 3C and 3D show additional exemplary results, similar to those shown in FIGS. 3A and 3B, obtained in a 28 year old healthy male, e.g., with positioning 312.

FIGS. 4A-4F show another sets of exemplary NC MRA images of abdominopelvic angiograms generated using the apparatus and method according to additional exemplary embodiments of the present disclosure, which provide the results obtained in a 51 year old volunteer who presented with scoliosis. In this exemplary complex geometry case, when a 20 mm thick SS-IR pulse 410 is oriented in oblique sagittal plane (see FIG. 4A) for a reinversion of the aorta, arterial coverage can be limited to common iliac arteries with TI of 1300 ms (see FIG. 4B). The arterial coverage can be extended to external iliac arteries with TI increased to 1500 ms (such as in FIG. 4C). When the thickness of the SS reinversion pulse was increased to 75 mm and oriented sagittally (see 420 and FIG. 4D), the arterial vessel conspicuity extended distally to the origin of the profunda femoris artery for both TI durations (e.g., TI of 1300 ms illustrated in FIG. 4E and TI of 1500 ms illustrated in FIG. 4F). However, by increasing the thickness of the SS re-inversion pulse, the signal intensity of the vena cava and the background within the SS pulse can be increased, resulting in reduced contrast between left renal artery and kidney due to the reinversion of kidney cortex.

The exemplary scan time of respiratory triggered NC MRA depended on each individual subject's breathing pattern. The exemplary mean scan time over all five subjects was 7.0±2.3 minutes.

FIGS. 5A and 5B show exemplary angiograms of a 66 year old patient using the exemplary sub-volume MIP of NC MRA and FIG. 5C shows an exemplary full volume MIP of subtraction CE MRA, generated with the apparatus and method according to exemplary embodiments of the present disclosure. As shown in FIGS. 5A to 5C, for example, both NC MRA and CE MRA exhibited depiction of infrarenal aortic atherosclerosis and left internal iliac artery origin stenosis. CR_{A-B}, achieved with NC can be about 0.92, comparable to subtraction CE MRA (e.g., CR_{A-B}=0.98) and higher than source CE MRA (e.g., CR_{A-B}=0.86). Increased background signal intensity, coincident with the location of the reinversion pulse 510 (e.g., as shown in FIG. 5A), can be observed with NC MRA in the vena cava 515, lymph nodes 520, bowel 525, and bladder 530. Anterior-posterior coverage of NC MRA can be smaller than that of CE MRA causing the truncated appearance of the internal iliac arteries. NC MRA can depict mild infrarenal aortic atherosclerosis and left internal iliac artery origin stenosis in agreement with CE MRA.

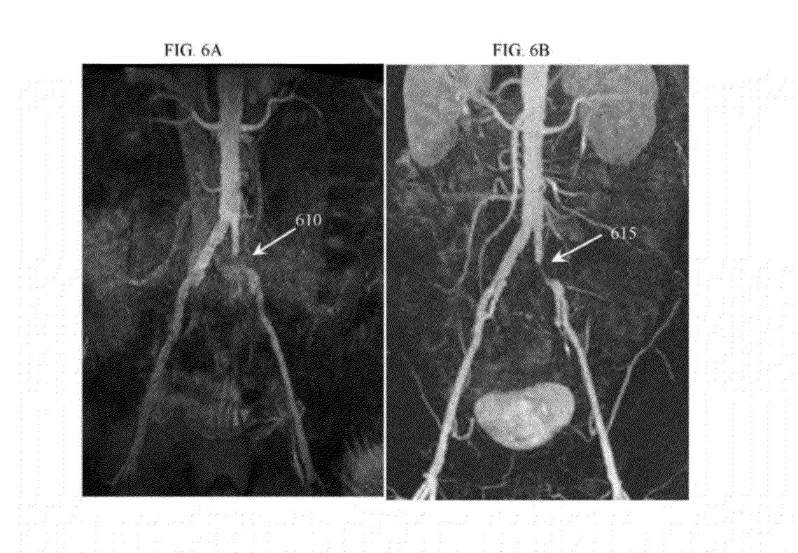

FIG. 6A shows an exemplary NC MRA image of a 66 year old male patient, imaged with TI=1300 ms, NC MRA, demonstrated complete visualization of the left iliac segment, distal to severe common iliac stenosis (see 610), which was confirmed by CE MRA, e.g., as shown in FIG. 6B at 615. An overall image quality of NC MRA was undermined, for example, due to anterior-posterior aliasing.

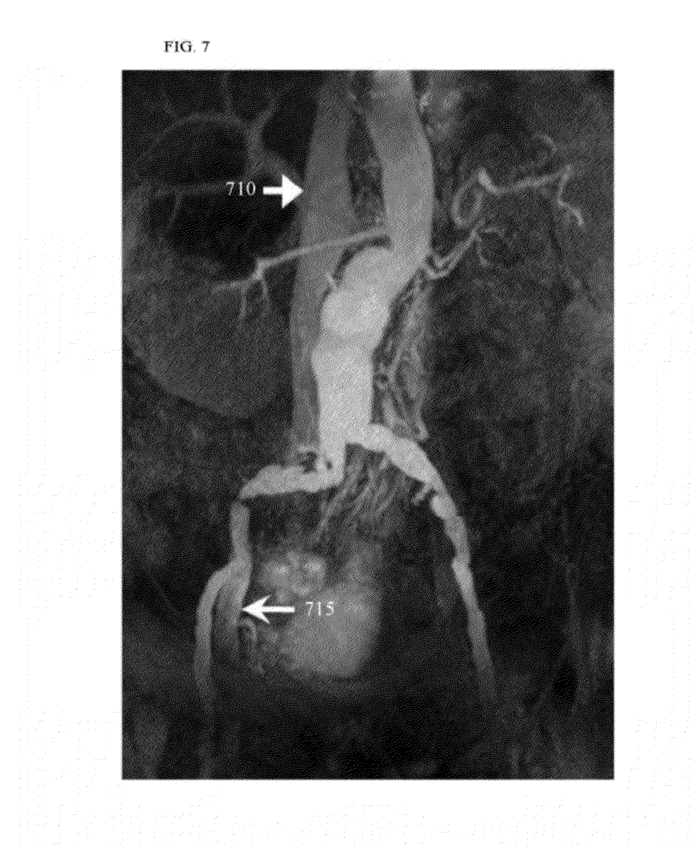

FIG. 7 shows an exemplary NC MRA image of an 80 year old male patient, with 96% of the length of the iliac arteries being visualized distal to aortic aneurysm with prolonged TI of 1700 ms. The thickness of the SS-IR pulse was extended, for example, to overlie the aneurysm at the expense of near complete reinversion of the vena cava and partial reinversion of the right external iliac vein (e.g., 710 and 715). For this patient CE MRA is not shown.

### Exemplary Image Quality Assessment

Exemplary Qualitative Assessment

In an exemplary implementation/utilization according to certain exemplary embodiments of the present disclosure, data can be de-identified and randomized for blinded review, for example, by two radiologists with 4 and 5 years MRA experience who can independently score the images. Source images can he used for both NC MRA and CE MRA evaluation. Eight vascular segments per subject (suprarenal artery, right and left renal artery, infrarenal artery, right and left common iliac, right and left external iliac) were assessed for diagnostic quality on a 4-point scale (0=non-diagnostic, 1=partially evaluable, 2=mostly evaluable, 3=fully evaluable). Presence of artifacts can be recorded on per segment basis. Each image can receive an overall diagnostic-quality score based on the same scale.

Exemplary Quantitative Assessment

In an exemplary implementation according to certain exemplary embodiments of the present disclosure, prior to quantitative assessment, 5 mm thick axial reconstructions of the 3D data sets can be obtained on an independent workstation (Leonardo, Siemens Healthcare). Region-of-interest (ROI) analysis can be performed on 12 of the resultant slices, grouped in 4 sets of 3, representative of the following arterial segments: suprarenal, infrarenal, common iliac, and external iliac. Arterial (S_{A}) and venous (S_{V}) signal intensities can be estimated as the mean intensity of all pixels contained within ROIs drawn to include the entire vessel lumen. Common and external iliac S_{A} and S_{V} can be calculated by averaging the intensities of the left and the right branches. ROIs for background signal (S_{B}) estimation can be placed over the right lobe of the liver for suprarenal slices and over the iliopsoas muscle for all remaining segments.

Relative signal contrast ratio between artery and background muscle, CR_{A-B} (=(S_{A}−S_{B})/S_{A}), as well as between artery and vein, CR_{A-V} (=(S_{A}−S_{V})/S_{A}), can be estimated for source NC MRA images and for source and subtraction CE MRA images. To evaluate the efficacy of venous suppression, vein-to-background contrast, CR_{V-B} (S_{V}−S_{B})/S_{A}), can also be measured. Mean CR_{A-B}, CR_{A-V}, and CR_{V-B} across the patients were calculated for the above-identified segments; individual segment measurements were averaged to obtain overall contrast ratios for the entire image.

Exemplary Craniocaudal Arterial Coverage

Maximal visible length of the common and external iliac arteries obtained with NC MRA can be measured as a fraction of total “Iliac” length. The latter can be defined from aortic bifurcation to origin of profunda femoris. For example, one radiologist can review the axial reconstruction images and record the last slice position in which the iliac segments were diagnostic. A visible length of the iliacs can be measured between this slice position and the aortic bifurcation. Longitudinal views of the iliac segments, obtained by curved multi-planar reconstructions, can be used for all measurements to control for differences in vessel geometry and orientation of the imaging slab.

### Exemplary Statistical Analysis

Diagnostic quality scores of NC MRA images obtained in patients can be compared to the reference standard (CE MRA) with a Wilcoxon signed rank test. A paired Student's t-test can be performed to determine the statistical significance of differences in C_{A-B}, C_{A-V}, and C_{V-B} between: a) source NC MRA and source CE MRA, and b) source NC MRA and subtraction CE MRA. In the tests, a P value<0.05 can be considered to be significant. Microsoft Office Excel version 12 can be used for the analyses.

### Exemplary Image Quality

In the seven volunteers overall image quality (see Table 1) averaged 2.79±0.39 on a scale of 0 to 3, where 3 is maximum. Using individual reader evaluations a total of 112 segments can be assessed, of which 86% (96/112) were rated fully evaluable (score=3). Fifteen (13%) segments received a score of 2 (mostly evaluable), occurring in the external iliac arteries (n=4) and the suprarenal aorta (n=11). The latter exhibited reduced signal intensity, likely caused by rapid flow of blood through an off-resonance region. One (1%) segment, part of an external iliac artery, can be judged partially evaluable due to insufficient inflow of unsaturated blood.

Artery-to-background contrast, CR_{A-B}, obtained in volunteers (see Table 2) averaged 0.90±0.03 with little variation across segments. Net CR_{A-V} and CR_{V-B} were 0.67±0.12 and 0.23±0.10, respectively, signifying adequate venous suppression.

TABLE 1
Image quality ratings of NC MRA in 7 controls and NC MRA
and CE MRA in 10 patients.

| | NC MRA | NC MRA | CE MRA |
|---|---|---|---|
| Arterial Segment | (volunteers) | (patients) | (patients) |
| Overall image quality | 2.79 ± 0.39 | 2.65 ± 0.41 | 2.90 ± 0.32 |
| Suprarenal artery | 2.50 ± 0.41 | 2.50 ± 0.39 | 2.90 ± 0.32 |
| Left renal artery | 3.00 ± 0.00 | 2.82 ± 0.40 | 2.90 ± 0.32 |
| Right renal artery | 2.93 ± 0.19 | 2.82 ± 0.40 | 2.90 ± 0.32 |
| Infrarenal artery | 3.00 ± 0.00 | 3.00 ± 0.00 | 2.90 ± 0.32 |
| Left com iliac artery | 2.93 ± 0.19 | 2.77 ± 0.47 | 2.94 ± 0.17 |
| Right com iliac artery | 2.93 ± 0.19 | 3.00 ± 0.00 | 3.00 ± 0.00 |
| Left ext iliac artery | 2.71 ± 0.57 | 2.59 ± 0.49* | 3.00 ± 0.00* |
| Right ext iliac artery | 2.78 ± 0.39 | 2.50 ± 0.55* | 3.00 ± 0.00* |

*p < 0.05

TABLE 2
Contrast ratios for NC MRA in volunteers (n = 7) and NC
MRA and CE MRA in patients (n = 10).

| | | NC MRA | NC MRA | CE MRA | CE MRA |
|---|---|---|---|---|---|
| Arterial Segment | | volunteers | patients | (source) | (subtraction) |
| Overall | CR_{A-B} | 0.90 ± 0.03 | 0.84 ± 0.06 | 0.82 ± 0.04 | 0.97 ± 0.01 |
| | CR_{A-V} | 0.67 ± 0.12 | 0.55 ± 0.17 | 0.75 ± 0.10 | 0.95 ± 0.03 |
| | CR_{V-B} | 0.23 ± 0.10 | 0.29 ± 0.13 | 0.10 ± 0.06 | 0.02 ± 0.02 |
| Suprarenal a. | CR_{A-B} | 0.84 ± 0.06 | 0.78 ± 0.13 | 0.77 ± 0.09 | 0.97 ± 0.01 |
| | CR_{A-V} | 0.65 ± 0.14 | 0.51 ± 0.19 | 0.75 ± 0.17 | 0.94 ± 0.06 |
| | CR_{V-B} | 0.19 ± 0.12 | 0.27 ± 0.17 | 0.07 ± 0.07 | 0.04 ± 0.06 |
| Infrarenal a. | CR_{A-B} | 0.90 ± 0.03 | 0.86 ± 0.06 | 0.85 ± 0.06 | 0.96 ± 0.02 |
| | CR_{A-V} | 0.60 ± 0.14 | 0.55 ± 0.16 | 0.75 ± 0.15 | 0.95 ± 0.05 |
| | CR_{V-B} | 0.30 ± 0.30 | 0.31 ± 0.12 | 0.10 ± 0.11 | 0.03 ± 0.04 |
| Common iliac a. | CR_{A-B} | 0.92 ± 0.02 | 0.86 ± 0.06 | 0.82 ± 0.08 | 0.97 ± 0.02 |
| | CR_{A-V} | 0.65 ± 0.15 | 0.56 ± 0.20 | 0.73 ± 0.15 | 0.96 ± 0.03 |
| | CR_{V-B} | 0.26 ± 0.14 | 0.30 ± 0.15 | 0.10 ± 0.08 | 0.01 ± 0.01 |
| External iliac a. | CR_{A-B} | 0.91 ± 0.02 | 0.84 ± 0.07 | 0.80 ± 0.08 | 0.98 ± 0.01 |
| | CR_{A-V} | 0.77 ± 0.10 | 0.58 ± 0.24 | 0.66 ± 0.17 | 0.96 ± 0.02 |
| | CR_{V-B} | 0.14 ± 0.08 | 0.26 ± 0.20 | 0.14 ± 0.11 | 0.02 ± 0.02 |

Exemplary Patients

In the exemplary ten patients, 82% (131/160) of the reviewed segments can be rated as fully evaluable, while 24 (15%) received a score of 2. Five segments (3%), encountered in the common (n=1) and external (n=4) iliac arteries, can be scored as partially evaluable. None were non-diagnostic. Overall image quality score of NC MRA (2.65±0.41) can be comparable to that of CE MRA (2.9±0.32) with no statistically significant difference observed between the two (p>0.2). When image quality scores of NC MRA and CE MRA were compared on per segment basis (see Table 1), statistically significant differences were found, for example, for the left and right external iliac arteries, with readers describing the following artifacts: insufficient inflow (n=1) and air-filled bowel peristalsis (n=2).

Overall artery-to-background contrast, CR_{A-B}, with NC MRA can average 0.84±0.06, 14% lower than CR_{A-B} of subtraction CE MRA. However, CR_{A-B} of the non-contrast technique can be comparable to that of source CE MRA with no statistically significant difference observed between the two (see Table 2). CR_{A-V} of NC MRA averaged 0.55±0.17 and can be significantly lower than CR_{A-V} of both source and subtraction CE MRA.

FIG. 5B shows a representative NC MRA image with the maximum image quality score obtained in a 66 year old patient. An overall artery-to-background signal contrast, CR_{A-B}=0.92, can be comparable to, for example, subtraction (CR_{A-B}=0.98) and source (CR_{A-B}=0.86) CE MRA. Venous signal throughout the image FIG. 5B shows a representative NC MRA image with the maximum image quality score obtained in a 66 year old patient. An overall artery-to-background signal contrast, for example, CR_{A-B}=0.92, can be comparable to subtraction (CR_{A-B}=0.98) and source (CR_{A-B}=0.86) CE MRA. Venous signal throughout the image can be deemed low by both readers, signifying adequate venous suppression, confirmed by contrast ratio measurements (CR_{A-V}=0.78, CR_{V-B}=0.13). However, medium to high venous signal can be observed in a portion of the vena cava due to the sagittal SS-IR pulse. Increased signal intensity, coincident with the location of the sagittal re-inversion pulse, was also encountered in lymph nodes, bladder, and bowel tissue.

### Exemplary Craniocaudal Coverage

Exemplary craniocaudal coverage obtained with exemplary NC MRA is summarized in Table 3. For example, in volunteers, a visualized arterial length distal to the bifurcation can average about 209-218 mm. Similar inflow distances can be observed in patients, corresponding to optimal visualization of 93-96% of the full length of the iliac arteries. In patients, similar coverage can be observed for the different TI values used (1300 and 1700 ms) likely due to variation of disease severity and cardiac output between the two groups.

TABLE 3
Mean craniocaudal coverage of arterial inflow obtained with NC
MRA volunteers (n = 7) and patients (n = 10)*

| | Iliac | Total visible | % visible | Iliac | Total visible | % visible |
|---|---|---|---|---|---|---|
| Subject | length | distance | iliac segment | length | distance | iliac segment |
| Group | (right) | (right) | (right) | (left) | (left) | (left) |
| Volunteers | 217 ± 8 | 218 ± 34 | 94 ± 8% | 205 ± 16 | 209 ± 48 | 93 ± 10% |
| (TI: 1300 ms) | | | | | | |
| Patients | 212 ± 19 | 202 ± 25 | 96 ± 5% | 211 ± 18 | 207 ± 55 | 93 ± 9% |
| (TI: 1300 ms) | | | | | | |
| Patients | 228 ± 38 | 217 ± 40 | 95 ± 4% | 216 ± 31 | 206 ± 32 | 95 ± 4% |
| (TI: 1700 ms) | | | | | | |

*Distance is measured in millimeters distal to the aortic bifurcation

For example, the left external iliac artery can be successfully completely visualized distal to severe stenosis of the common iliac artery in a 66 year old patient imaged with TI=1300 ms (see FIGS. 6A and 6B). In an 80 year old patient (see FIG. 7), near complete (96%) depiction of bilateral iliac arteries can be obtained distal to an aortic aneurysm by extending TI to 1700 ms and broadening the SS-IR pulse to completely cover the aneurysm. Suboptimal inflow can occur in one volunteer (visible left iliac 75%, visible right iliac 77%) and one patient without pathology (visible left iliac 82%, visible right iliac 91%) both imaged with TI 1300 ms.

Vascular pathology can be present, for example, in five patients. Left and right renal artery stenosis, mild infrarenal atherosclerosis, and infrarenal aortic aneurysm can be identified by NC MRA in agreement with CE MRA findings.

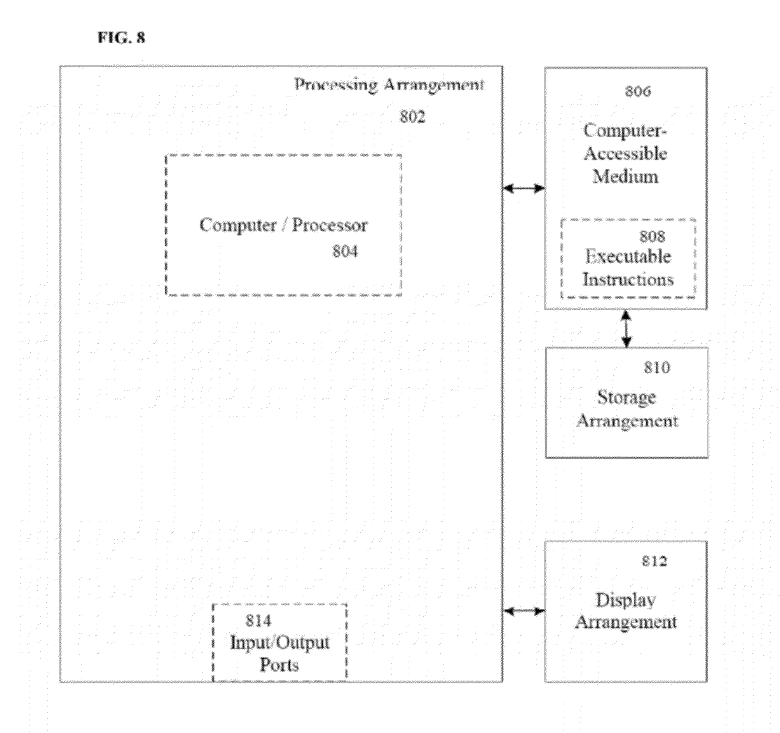

FIG. 8 shoes an exemplary block diagram of an exemplary embodiment of a system according to the present disclosure. For example, exemplary procedures in accordance with the present disclosure described herein can be performed by a processing arrangement and/or a computing arrangement 802. Such processing/computing arrangement 802 can be, e.g., entirely or a part of, or include, but not limited to, a computer/processor 804 that can include, e.g., one or more microprocessors, and use instructions stored on a computer-accessible medium (e.g., RAM, ROM, hard drive, or other storage device),

As shown in FIG. 8, e.g., a computer-accessible medium 806 (e.g., as described herein above, a storage device such as a hard disk, floppy disk, memory stick, CD-ROM, RAM, ROM, etc., or a collection thereof) can be provided (e.g., in communication with the processing arrangement 802). The computer-accessible medium 806 can contain executable instructions 808 thereon. In addition or alternatively, a storage arrangement 810 can be provided separately from the computer-accessible medium 806, which can provide the instructions to the processing arrangement 802 so as to configure the processing arrangement to execute certain exemplary procedures, processes and methods, as described herein above, for example.

Further, the exemplary processing arrangement 802 can be provided with or include an input/output arrangement 814, which can include, e.g., a wired network, a wireless network, the internet, an intranet, a data collection probe, a sensor, etc. As shown in FIG. 8, the exemplary processing arrangement 802 can be in communication with an exemplary display arrangement 812, which, according to certain exemplary embodiments of the present disclosure, can be a touch-screen configured for inputting information to the processing arrangement in addition to outputting information from the processing arrangement, for example. Further, the exemplary display 812 and/or a storage arrangement 810 can be used to display and/or store data in a user-accessible format and/or user-readable format.

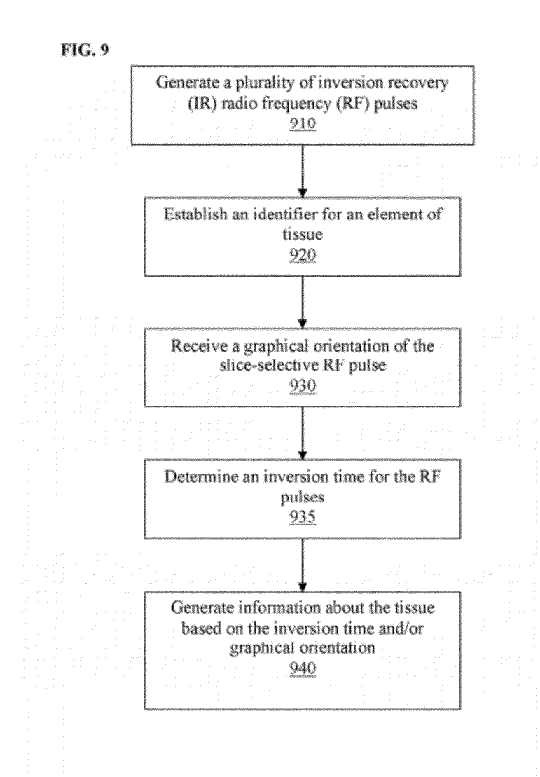

FIG. 9 shows an exemplary flow diagram of an exemplary procedure according to an exemplary embodiment of the present disclosure. As illustrated in FIG. 9, the exemplary procedure can generate (e.g., with a computing arrangement, such as a hardware processor and a source) a plurality of inversion recovery (IR) radio frequency (RF) pulses at block 910. An exemplary RF pulse can include at least one nonselective IR RF pulse that can be configured to invert a longitudinal magnetization of at least one background tissue. These pulses can establish an identifier for an element of tissue, e.g., at block 920, such as blood traveling through an aortic bifurcation. Establishing the identifier (e.g., as in block 920), can include re-inverting a longitudinal magnetization of the element associated with the tissue.

The RF pulses can also include a graphically oriented slice-selective RF pulse configured to re-invert the longitudinal magnetization of magnetic spins of the element associated with the tissue. At block 930, the exemplary procedure can receive a graphical orientation of the graphically oriented slice-selective RF pulse. The graphical orientation can facilitate focusing on the tissue and excluding other tissues, e.g., background tissues. The RF pulses can also include a graphically oriented further slice-selective RF pulse configured to invert a longitudinal magnetization of an additional tissue.

At block 935, the exemplary procedure can determine an inversion time for one or more RF pulses. The exemplary RF pulses can include a short inversion time inversion recovery (STIR) RF pulse, which can be configured to invert a longitudinal magnetization of at least some of the tissues within an imaging volume. The exemplary the RF pulses can include a graphically oriented slice-selective RF pulse, which can be configured to re-invert the longitudinal magnetization of magnetic spins of the element associated with the at least one tissue immediately following a first nonselective RF pulse, which itself can be configured to invert a longitudinal magnetization of background tissues.

At 9 block 40, the exemplary procedure can generate information (e.g., an enhanced graphical representation, enhanced image volume, or another number of other output metrics), about the tissue (e.g., the target tissue of interest). This information can be based on the inversion time determined in 935 and/or the graphical orientation received (or determined) in 930, along with any number of other inputs.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly incorporated herein in its entirety. All publications referenced above can be incorporated herein by reference in their entireties.

What is claimed is:

1. A method for generating information regarding at least one tissue, comprising:
generating a plurality of inversion recovery (IR) radio frequency (RF) pulses that are configured to establish an identifier of an element associated with at least one portion of the at least one tissue;
determining an inversion time associated with at least one of the RF pulses; and
generating, with a computing arrangement, the information regarding the at least one tissue based on the inversion time.

2. The method of claim 1, wherein the at least one tissue includes an aortic bifurcation and the element includes blood traveling through the aortic bifurcation.

3. The method of claim 1, wherein the RF pulses include at least one first nonselective IR RF pulse configured to invert a longitudinal magnetization of at least one background tissue.

4. The method of claim 1, wherein establishing the identifier includes re-inverting a longitudinal magnetization of the element associated with the at least one tissue.

5. The method of claim 4, wherein the RF pulses include a graphically oriented slice-selective RF pulse configured to re-invert the longitudinal magnetization of magnetic spins of the element associated with the at least one tissue.

6. The method of claim 5, further comprising receiving a graphical orientation of the graphically oriented slice-selective RF pulse, wherein the information is generated using the graphical orientation.

7. The method of claim 5, wherein the graphical orientation facilitates substantially focusing on the at least one tissue and exclude background tissues.

8. The method of claim 5, wherein the RF pulses include a graphically oriented further slice-selective RF pulse configured to invert a longitudinal magnetization of an additional tissue.

9. The method of claim 8, further comprising receiving a graphical orientation of the graphically oriented further slice-selective RF pulse, wherein the information is generated using the graphical orientation of the further slice-selective RF pulse.

10. The method of claim 1, wherein the RF pulses include a short inversion time inversion recovery (STIR) RF pulse configured to invert a longitudinal magnetization of at least some tissues within an imaging volume.

11. The method of claim 1, wherein the RF pulses include a graphically oriented slice-selective RF pulse configured to re-invert the longitudinal magnetization of magnetic spins of the element associated with the at least one tissue immediately following a first nonselective RF pulse which is configured to invert a longitudinal magnetization of background tissues.

12. The method of claim 1, wherein the at least one tissue includes aortic bifurcation.

13. The method of claim 1, wherein the RF pulses are IR pulses.

14. A non-transitory computer readable medium including instructions thereon that are accessible by a hardware processing arrangement, wherein, when the processing arrangement executes the instructions, the processing arrangement is configured to:
generate a plurality of inversion recovery (IR) radio frequency (RF) pulses that are configured to establish an identifier of an element associated with at least one portion of the at least one tissue;
determine an inversion time associated with at least one of the pulses; and
generate the information regarding the at least one tissue based on the inversion time.

15. The non-transitory computer readable medium of claim 14, wherein the at least one tissue includes an aortic bifurcation and the element includes blood traveling through the aortic bifurcation.

16. The computer readable medium of claim 14, wherein the RF pulses include a graphically oriented slice-selective RF pulse configured to re-invert the longitudinal magnetization of magnetic spins of the element associated with the at least one tissue immediately following a first nonselective RF pulse which is configured to invert a longitudinal magnetization of background tissues.

17. The computer readable medium of claim 14, wherein establishing the identifier includes reinverting a longitudinal magnetization of the element associated with the at least one tissue.

18. The computer readable medium of claim 14, further comprising graphically orienting the slice selective RF pulse to substantially focus on the at least one tissue and exclude background tissues.

19. An imaging apparatus for obtaining information regarding at least one tissue, comprising:
a computer readable medium which includes instructions thereon that are accessible by a hardware processing arrangement, wherein, when the processing arrangement executes the instructions, the processing arrangement is configured to:
generate a plurality of radio frequency (RF) pulses that are configured to establish an identifier of an element associated with at least one portion of the at least one tissue;
determine an inversion time associated with at least one of the pulses; and
generate the information regarding the at least one tissue based on the inversion time.

20. The apparatus of claim 19, wherein the RF pulses include a graphically oriented slice-selective RF pulse configured to re-invert the longitudinal magnetization of magnetic spins of the element associated with the at least one tissue immediately following a first nonselective RF pulse which is configured to invert a longitudinal magnetization of background tissues.

21. The apparatus of claim 19, wherein establishing the identifier includes reinverting longitudinal magnetization of the element associated with at least one:portion of the at least one tissue.

22. The apparatus of claim 19, further comprising graphically orienting the slice selective RF pulse to substantially focus on the at least one tissue and exclude background tissues.
